(19) United States
(12) Patent Application Publication
(10) Pub. No.: US 2009/0221576 A1
(43) Pub. Date: Sep. 3, 2009

(54) Pharmaceutical Use of 2-Hydroxy-3-[5-(Morpholin-4-Ylmethyl) Pyridin-2-YL]-1H-IN-Dole-5-Carbonitrile as a Free Base or Salts

(75) Inventors: Anna-Lena Berg, Sodertalje (SE); Ratan Bhat, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB GLOBAL INTELLECTUAL PROPERTY, SODERTALJE (SE)

(21) Appl. No.: 12/162,547

(22) PCT Filed: Jan. 31, 2007
(86) PCT No.: PCT/SE07/00087

§ 371 Date: Jul. 29, 2008

Related U.S. Application Data
(60) Provisional application No. 60/777,348, filed on Feb. 28, 2006.
(60) Provisional application No. 60/764,551, filed on Feb. 2, 2006.

(51) Int. Cl. A61K 31/5377

(52) U.S. Cl. 514/235.2

(57) ABSTRACT

New use of 2-hydroxy-3-[5-(morpholin-4-ylmethyl)pyridin-2-yl]1H-indole-5-carbonitrile as a free base or a pharmaceutically acceptable salt thereof, in a method of prevention and/or treatment of bone-related disorders, osteoporosis, increasing bone formation or increasing bone mineral density.

### FIELD OF THE INVENTION

The present invention relates to a new use of certain GSK3 inhibitors, namely 2-hydroxy-3-[5-(morpholin-4-ylmethyl)pyridin-2-yl]1H-indole-5-carbonitrile in the manufacture of a medicament for the treatment and/or prevention of bone-related disorders or conditions, such as osteoporosis and increased bone formation and bone mineral density. The present invention further relates to a method of treatment and/or prevention of these disorders or conditions.

### BACKGROUND OF THE INVENTION

Glycogen synthase kinase 3 (GSK3) is a serine/threonine protein kinase composed of two isoforms (α and β), which are encoded by distinct genes but are highly homologous within the catalytic domain. GSK3 is highly expressed in the central and peripheral nervous system. GSK3 phosphorylates several substrates including tau, β-catenin, glycogen synthase, pyruvate dehydrogenase and elongation initiation factor 2b (eIF2b). Insulin and growth factors activate protein kinase B, which phosphorylates GSK3 on the serine 9 residue and inactivates it.

### GSK3 and Bone Disorders

Remodeling of the skeleton is a continuous process, controlled by systemic hormones such as parathyroid hormone (PTH), local factors (e.g. prostaglandin E₂), cytokines and other biologically active substances. Two cell types are of key importance: osteoblasts (responsible for bone formation) and osteoclasts (responsible for bone resorption). Via the RANK, RANK ligand and osteoprotegerin regulatory system these two cell types interact to maintain normal bone turnover (Bell N H, Current Drug Targets—Immune, Endocrine & Metabolic Disorders, 2001, 1:93-102).

Osteoporosis is a skeletal disorder in which low bone mass and deterioration of bone microarchitecture lead to increased bone fragility and fracture risk. To treat osteoporosis, the two main strategies are to either inhibit bone resorption or to stimulate bone formation. The majority of drugs currently on the market for the treatment of osteoporosis act to increase bone mass by inhibiting osteoclastic bone resorption. It is recognized that a drug with the capacity to increase bone formation would be of great value in the treatment of osteoporosis as well as having the potential to enhance fracture healing in patients.

Recent in vitro studies suggest a role of GSK3β in osteoblast differentiation. First, it has been shown that glucocorticoids inhibit cell cycle progression during osteoblast differentiation in culture. The mechanism behind this is activation of GSK3β in osteoblasts, resulting in c-Myc down-regulation and impediment of the G₁/S cell cycle transition. The attenuated cell cycle and reduced c-Myc level are returned to normal when GSK3β is inhibited using lithium chloride (Smith et al., J. Biol. Chem., 2002, 277:18191-18197). Secondly, inhibition of GSK3β in the pluripotent mesenchymal cell line C3H10T1/2 leads to a significant increase in endogenous β-catenin signaling activity. This, in turn, induces expression of alkaline phosphatase mRNA and protein, a marker of early osteoblast differentiation (Bain et al., Biochem. Biophys. Res. Commun., 2003, 301:84-91).

Published in vivo data confirming the in vitro effects of GSK3β on osteoblast differentiation are still lacking. However, studies by the inventors clearly show an increased bone formation in rats treated with a GSK3β inhibitor (see below under Examples). It should also be noted that patients treated with lithium have increased levels of bone-specific alkaline phosphatase, indirectly providing support for the notion that inhibition of GSK3β would lead to osteoblast stimulation and increased bone formation (Broulik et al., Clinica Chemica Acta, 1984, 140:151-155).

### DETAILED DESCRIPTION OF THE INVENTION

The present invention is directed to the use of 2-hydroxy-3-[5-(morpholin-4-ylmethyl)pyridin-2-yl]1H-indole-5-carbonitrile (compound (I))

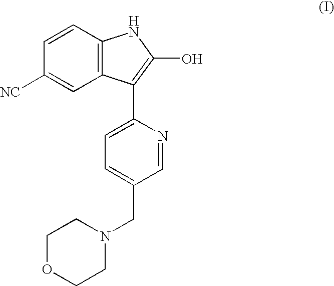

as a free base or a pharmaceutically acceptable salt thereof, for the manufacture of a medicament for use in the treatment and/or prevention of bone-related disorders or conditions.

A further aspect of the invention relates to the use of the following pharmaceutically acceptable salts of the compound of the formula (I), namely
2-hydroxy-3-[5-(morpholin-4-ylmethyl)pyridin-2-yl]1H-indole-5-carbonitrile citrate,
2-hydroxy-3-[5-(morpholin-4-ylmethyl)pyridin-2-yl]1H-indole-5-carbonitrile mesylate,
2-hydroxy-3-[5-(morpholin-4-ylmethyl)pyridin-2-yl]1H-indole-5-carbonitrile esylate,
2-hydroxy-3-[5-(morpholin-4-ylmethyl)pyridin-2-yl]1H-indole-5-carbonitrile edisylate,
2-hydroxy-3-[5-(morpholin-4-ylmethyl)pyridin-2-yl]1H-indole-5-carbonitrile phosphate,
2-hydroxy-3-[5-(morpholin-4-ylmethyl)pyridin-2-yl]1H-indole-5-carbonitrile fumarate and
2-hydroxy-3-[5-(morpholin-4-ylmethyl)pyridin-2-yl]1H-indole-5-carbonitrile maleate for the manufacture of a medicament for use in the treatment and/or prevention of bone-related disorders or conditions.

One aspect of the invention is directed to the use of a compound of the formula (I), as a free base or a pharmaceutically acceptable salt thereof, to treat osteoporosis.

One aspect of the invention is directed to the use of a compound of the formula (I), as a free base or a pharmaceutically acceptable salt thereof, to increase and promote bone formation in mammals.

One aspect of the invention is directed to the use of a compound of the formula (I), as a free base or a pharmaceutically acceptable salt thereof, to increase bone mineral density in mammals.

Another aspect of the invention is directed to the use of a compound of the formula (I), as a free base or a pharmaceutically acceptable salt thereof, to reduce the rate of fracture and/or increase the rate of fracture healing in mammals.

Another aspect of the invention is directed to the use of a compound of the formula (I), as a free base or a pharmaceutically acceptable salt thereof, to increase cancellous bone formation and/or new bone formation in mammals.

Another aspect of the invention is directed to a method of prevention and/or treatment of bone-related disorders comprising administering to a mammal in need of such prevention and/or treatment, a therapeutically effective amount of a compound of the formula (I) as a free base or a pharmaceutically acceptable salt thereof.

Another aspect of the invention is directed to a method of prevention and/or treatment of osteoporosis comprising administering to a mammal in need of such prevention and/or treatment, a therapeutically effective amount of a compound of the formula (I) as a free base or a pharmaceutically acceptable salt thereof.

Another aspect of the invention is directed to a method of increasing bone formation comprising administering to a mammal in need of such treatment, a therapeutically effective amount of a compound of the formula (I) as a free base or a pharmaceutically acceptable salt thereof.

Another aspect of the invention is directed to a method of increasing bone mineral density comprising administering to a mammal in need of such treatment, a therapeutically effective amount of a compound of the formula (I) as a free base or a pharmaceutically acceptable salt thereof.

Another aspect of the invention is directed to a method of reducing the incidence of fracture comprising administering to a mammal in need of such treatment, a therapeutically effective amount of a compound of the formula (I) as a free base or a pharmaceutically acceptable salt thereof.

Another aspect of the invention is directed to a method of enhancing fracture healing comprising administering to a mammal in need of such treatment, a therapeutically effective amount of a compound of the formula (I) as a free base or a pharmaceutically acceptable salt thereof.

Another aspect of the invention is directed to said methods and wherein said mammal is a human.

Another aspect of the invention is directed to said methods and wherein said mammal is an animal, preferably but not limited to bigger animals such as horses, camels, dromedars but not limited thereto.

2-Hydroxy-3-[5-(morpholin-4-ylmethyl)pyridin-2-yl]1H-indole-5-carbonitrile as a free base or a pharmaceutically acceptable salt thereof are disclosed in WO03/082853. The effect of the compound (I) of the present invention on bone growth has been investigated. It has been found that such a compound are well suited to promote and increase bone formation, increase bone mineral density and consequently for inhibiting bone-related disorders such as osteoporosis.

The use of the GSK3 inhibitors, the compounds of formula (I), in primary and secondary osteoporosis, where primary osteoporosis includes postmenopausal osteoporosis and senile osteoporosis in both men and women, and secondary osteoporosis includes cortison induced osteoporosis, as well as any other type of induced secondary osteoporosis, are included in the term osteoporosis. In addition to this, these GSK3 inhibitors may also be used in treatments of myeloma. These GSK3 inhibitors may be administered locally or systemically, in different formulation regimes, to treat these conditions.

The promotion and increasing of bone formation makes the compounds of the formula (I) suitable to reducing the incidence of fracture, to reduce the rate of fracture and/or increase the rate of fracture healing, to increase cancellous bone formation and/or new bone formation in mammals.

The use to promote and increase new bone formation may be in connection with surgery. This invention can be used during surgery, where the treating surgeon will place the invention locally in an appropriate formulation, near the deficient bone and/or in the body cavity. The bone may for instance have been broken, and utilizing the invention as described and claimed herein will then be placed in or near the fracture during open fracture repair. In some instances bone pieces may be missing (e.g. after tumour removal or severe casualties), and utilizing the invention as described and claimed herein will then be placed near the site of constructive bone surgery.

A suitable pharmaceutically acceptable salt of the compound useful in accordance to the invention is, for example, an acid-addition salt, which is sufficiently basic, for example an inorganic or organic acid. In addition a suitable pharmaceutically acceptable salt of the compounds of the invention, which is sufficiently acidic, is an alkali metal salt, an alkaline earth metal salt or a salt with an organic base, which affords a physiologically-acceptable cation.

The compound of the formula (I) or salt thereof, may be prepared as described in WO03/082853, which hereby is incorporated by reference.

The formation of the desired salt of the compound of the formula (I), 2-hydroxy-3-[5-(morpholin-4-ylmethyl)pyridin-2-yl]1H-indole-5-carbonitrile citrate, mesylate, esylate, edisylate, phosphate, fumarate or maleate, may be prepared by mixing 2-hydroxy-3-[5-(morpholin-4-ylmethyl)pyridin-2-yl]1H-indole-5-carbonitrile with the appropriate acid in the presence of a solvent. The equivalent of the appropriate acid may vary between 0.5 and 1 mole equivalents. The reaction may be performed in a solvent, suitable solvents are ethers such as 1,4-dioxane, diethyl ether or alcohols such as methanol, ethanol, propanol, or ketones such as acetone, isobutylmethylketone, or acetates such as ethyl acetate, butylacetate, or organic acids such as acetic acid, or water, or mixtures thereof. The total volume of solvents used may vary between 1 (v/w) to 100 (v/w) volume parts per weight of starting material, preferably between 10 (v/w) and 45 (v/w) volume parts per weight of starting material. The temperature of the reaction may be between −30 and 150° C., preferably between −5° C. and 100° C.

Pure compound of the formula (I), 2-hydroxy-3-[5-(morpholin-4-ylmethyl)pyridin-2-yl]1H-indole-5-carbonitrile citrate, mesylate, esylate, edisylate, phosphate, fumarate or maleate, may be obtained by crystallising with or without an additive in suitable solvents to obtain a crystalline solid having a purity of about 95% and preferably about 98%

### Pharmaceutical Composition

The composition used in accordance with the present invention may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration as an ointment, patch or cream, for rectal administration as a suppository and for local administration in a body cavity or in a bone cavity.

Suitable daily doses of the compounds of the formula (I) used in the treatment of a mammal, including human, are approximately from 0.01 to 250 mg/kg bodyweight at peroral administration and from about 0.001 to 250 mg/kg bodyweight at parenteral administration. The typical daily dose of the active ingredients varies within a wide range and will depend on various factors such as the relevant indication, the route of administration, the age, weight and sex of the patient and may be determined by a physician.

Illustrative representative pharmaceutical dosage forms containing the compound of the formula (I), 2-hydroxy-3-[5-(morpholin-4-ylmethyl)pyridin-2-yl]1H-indole-5-carbonitrile as free base or pharmaceutically acceptable salt thereof, including citrate, mesylate, esylate, edisylate, phosphate, fumarate or maleate, are described in WO03/082853, which dosage forms are hereby incorporated by reference.

For veterinary use the amounts of different components, the dosage form and the dose of the medicament may vary and will depend on various factors such as, for example the individual requirement of the animal treated.

It has been found that bone formation and bone mineral density can be increased by the uses of compounds of the formula (I) above. The term “therapy” as used in accordance with the invention also includes “prevention” unless there are specific indications to the contrary. The terms “therapeutic” and “therapeutically” should be construed accordingly.

### EXAMPLES

The following Examples 1 to 7 describes the preparation of the named specific salts of the compound (I), which are an aspect of the present invention.

### Example 1

### 2-Hydroxy-3-[5-(morpholin-4-ylmethyl)pyridin-2-yl]1H-indole-5-carbonitrile citrate salt

2-Hydroxy-3-[5-(morpholin-4-ylmethyl)pyridin-2-yl]1H-indole-5-carbonitrile (5.14 kg, 15.4 mol) was suspended in ethanol (54 L) at room temperature. The suspension was heated to an inner temperature of 70° C. and a solution of citric acid (3.424 kg, 17.82 mol) in water (103 L) was added keeping the inner temperature above 65° C. The mixture was heated to reflux. After this the resulting solution was mixed with activated charcoal (0.412 kg) and reflux continued for 3.5 h after which the reaction mixture was clear filtered at 83° C. followed by cooling to room temperature over 20 h. After filtration the precipitate was washed twice with a cold mixture of ethanol/water (6.9 L/13.7 L). Drying under vacuum at 50° C. gave 6.648 kg, 82.2% yield of 2-hydroxy-3-[5-(morpholin-4-ylmethyl)-pyridin-2-yl]1H-indole-5-carbonitrile citrate having a purity of at least 98%. The palladium content was less than 1 ppm and the zinc content was lower than 10 ppm. ¹H NMR (d6-DMSO, 400 MHz) δ 14.8 (br s, 1H), 10.98 (s, 1H), 8.1 (s 1H), 7.55 (m, 3H), 7.31 (d, 1H), 7.02 (d, 1H), 3.6 (s, 4H), 3.45 (m, 2H), 2.75 (ap d, 2H), 2.65 (ap d, 2H), 2.47 (s, 4H) ppm; ¹³C NMR (d6-DMSO, 400 MHz) δ 174.9, 171.3, 168.7, 148.4, 142.1, 137.1, 136.4, 125.2, 124.1, 121.1, 121.0, 118.8, 118.4, 101.4, 84.6, 72.3, 65.7, 58.0, 52.5, 42.9 ppm; MS (ES) m/z [M⁺+1] 335.

The crystals were analysed by X-ray powder diffraction (XRPD). The diffractogram of Form A shows the following d-values given in Angstrom and relative intensities: 12.7 (vs), 7.6 (w), 6.8 (vs), 6.3 (s), 5.9 (w), 5.7 (m), 5.1 (m), 4.87 (w), 4.57 (m), 4.38 (s), 4.23 (s), 4.16 (w), 4.07 (m), 3.80 (w), 3.69 (m), 3.65 (w), 3.41 (m), 3.37 (w), 3.32 (m), 3.17 (m), 3.12 (m), 2.88 (w), 2.86 (m), 2.78 (w), 2.65 (w), 2.50 (w), 2.45 (w).

The significant d-values given in Angstrom and relative intensities are: 12.7 (vs), 6.8 (vs), 6.3 (s), 4.38 (s), 4.23 (s), 3.41 (m).

The relative intensities (rel.int.) are less reliable and instead of numerical values the following definitions are used:

| | | |
|---|---|---|
| vs (very strong): | >60% | rel int. |
| s (strong): | 23-60% | rel int. |
| m (medium): | 9-23% | rel int. |
| w (weak): | 4-9% | rel int. |
| vw (very weak): | <4% | rel int. |

### Example 2

### 2-Hydroxy-3-[5-(morpholin-4-ylmethyl)pyridin-2-yl]1H-indole-5-carbonitrile mesylate

2-hydroxy-3-[5-(morpholin-4-ylmethyl)pyridin-2-yl]1H-indole-5-carbonitrile (0.6145 g) was suspended in ethanol (6.1 ml) and methane sulfonic acid (0.11 ml). The solution was heated to 40° C. for 14 hrs, and is then cooled to room temperature in 2 hrs. The crystals were filtered, washed with ethanol (2 ml) and dried in vacuum at 40° C. for 47 hrs. 0.6690 g 2-hydroxy-3-[5-(morpholin-4-ylmethyl)pyridin-2-yl]1H-indole-5-carbonitrile mesylate was obtained after drying.

¹H (500 MHz, DMSO-d₆): 14.75 (1H, br s), 10.97 (1H, s), 9.92 (1H, br s), 8.25 (1H, s), 8.00 (1H, s), 7.89 (2H, br m), 7.33 (1H, dd), 7.03 (1H, d), 4.30 (2H, br s), 3.98 (2H, br s), 3.66 (2H, br s), 3.37 (2H, br s), 3.12 (2H, br s), 2.38 (3H, s).

The crystals were analysed by X-ray powder diffraction (XRPD). The diffractogram shows the following d-values (given in angstrom) and relative intensities: 12.7 (vs), 9.0 (w), 7.3 (vw), 6.4 (s), 5.8 (vw), 5.7 (vw), 5.3 (w), 4.93 (w), 4.80 (m), 4.72 (m), 4.53 (m), 4.46 (m), 4.31 (m), 4.24 (m), 4.09 (s), 3.96 (m), 3.79 (m), 3.33 (s), 3.21 (m), 2.94 (w), 2.73 (w), 2.47 (w) Å.

The significant d-values (given in angstrom) and relative intensities are: 12.7 (vs), 6.4 (s), 4.53 (m), 4.09 (s), 3.33 (s) Å.

The relative intensities (% rel.int.) are less reliable and instead of numerical values the following definitions are used:

vs (very strong): >83
(strong): 60-83
m (medium): 17-60
w (weak): 7-17
vw (very weak): <7

### Example 3

### 2-Hydroxy-3-[5-(morpholin-4-ylmethyl)pyridin-2-yl]1H-indole-5-carbonitrile esylate

2-hydroxy-3-[5-(morpholin-4-ylmethyl)pyridin-2-yl]1H-indole-5-carbonitrile (0.5006 g) was suspended in ethanol (5 ml) and ethane sulfonic acid (0.12 ml). The solution was heated to 40° C. for 14 hrs, and was then cooled to room temperature in 2 hrs. The crystals were filtered, washed with ethanol (2 ml) and dried in vacuum at 40° C. for 47 hrs. 0.5487 g 2-hydroxy-3-[5-(morpholin-4-ylmethyl)pyridin-2-yl]1H-indole-5-carbonitrile esylate was obtained after drying.

¹H (500 MHz, DMSO-d₆): 14.72 (1H, br s), 10.98 (1H, s), 9.85 (1H, br s), 8.25 (1H, s), 8.00 (1H, s), 7.88 (2H, dd), 7.34 (1H, d), 7.03 (1H, d), 4.32 (2H, br s), 4.00 (2H, br d), 3.64 (2H, br t), 3.37 (2H, br s), 3.13 (2H, bd s), 2.40 (2H, q), 1.07 (3H, t).

The crystals were analysed by X-ray powder diffraction (XRPD). The diffractogram shows the following d-values (given in angstrom) and relative intensities: 15.3 (m), 12.6 (s), 9.1 (m), 7.6 (vw), 7.4 (m), 6.3 (m), 5.9 (w), 5.5 (w), 5.2 (vw), 5.0 (w), 4.87 (m), 4.74 (m), 4.47 (m), 4.32 (m), 4.16 (s), 4.12 (s), 4.04 (m), 3.85 (m), 3.41 (s), 3.19 (m), 2.92 (vw), 2.72 (w) Å.

The significant d-values (given in angstrom) and relative intensities are: 12.6 (s), 6.3 (m), 4.47 (m), 4.16 (s), 4.12 (s), 3.41 (s) Å.

The relative intensities (% rel.int.) are less reliable and instead of numerical values the following definitions are used:

(strong): >50
m (medium): 12-50
w (weak): 8-12
vw (very weak <8:

### Example 4

### 2-Hydroxy-3-[5-(morpholin-4-ylmethyl)pyridin-2-yl]1H-indole-5-carbonitrile edisylate

2-hydroxy-3-[5-(morpholin-4-ylmethyl)pyridin-2-yl]1H-indole-5-carbonitrile (0.6568 g) was suspended in ethanol (6.55 ml) and 1,2-ethane disulfonic acid (0.3471 g. The solution was heated to 40° C. for 14 hrs, and was then cooled to room temperature in 2 hrs. The crystals were filtered, washed with ethanol (2 ml) and dried in vacuum at 40° C. for 47 hrs. 0.7797 g 2-hydroxy-3-[5-(morpholin-4-ylmethyl)pyridin-2-yl]1H-indole-5-carbonitrile edisylate was obtained after drying.

¹H (500 MHz, DMSO-d₆): 14.74 (1H, br s), 10.98 (1H, s), 9.87 (1H, br s), 8.27 (1H, s), 8.00 (1H, d), 7.89 (2H, br m), 7.33 (1H, dd), 7.03 (1H, d), 4.33 (2H, s), 4.00 (2H, d), 3.65 (2H, t), 3.39 (2H, d), 3.14 (2H, br q), 2.74 (4H, s).

The crystals were analysed by X-ray powder diffraction (XRPD). The diffractogram shows the following d-values (given in angstrom) and relative intensities: 19.9 (m), 18.1 (w), 15.3 (s), 13.2 (vw), 11.3 (m), 9.0 (vw), 8.2 (w), 8.0 (w), 7.6 (w), 7.4 (w), 6.8 (w), 6.6 (m), 6.3 (m), 6.0 (w), 5.6 (s), 5.4 (m), 5.2 (m), 5.1 (m), 5.0 (m), 4.85 (m), 4.32 (s), 4.24 (m), 4.17 (w), 4.12 (s), 4.08 (s), 3.90 (w), 3.82 (w), 3.66 (w), 3.53 (m), 3.35 (m), 3.27 (m), 3.00 (vw) Å.

The significant d-values (given in angstrom) and relative intensities are: 15.3 (s), 11.3 (m), 5.6 (s), 4.32 (s), 4.12 (s), 4.08 (s) Å.

The relative intensities (% rel.int.) are less reliable and instead of numerical values the following definitions are used:

(strong): >55
m (medium): 24-55
w (weak): 13-24
vw (very weak): <13

### Example 5

### 2-Hydroxy-3-[5-(morpholin-4-ylmethyl) pyridin-2-yl]1H-indole-5-carbonitrile phosphate

2-hydroxy-3-[5-(morpholin-4-ylmethyl)pyridin-2-yl]1H-indole-5-carbonitrile (0.5919 g) was suspended in ethanol (5.9 ml) and o-phosphorous acid (0.1 ml. The solution was heated to 40° C. for 14 hrs, and was then cooled to room temperature in 2 hrs. The crystals were filtered, washed with ethanol (2 ml) and dried in vacuum at 40° C. for 47 hrs. 0.6666 g 2-hydroxy-3-[5-(morpholin-4-ylmethyl)pyridin-2-yl]1H-indole-5-carbonitrile phosphate was obtained after drying.

¹H (500 MHz, DMSO-d₆): 14.78 (1H, br s), 10.90 (1H, s), 8.12 (1H, s), 7.91 (1H, s), 7.85 (1H, br d), 7.81 (1H, dd), 7.29 (1H, dd), 7.01 (1, d), 3.60 (4H, br t), 3.46 (2H, s), 2.45 (4H, br s).

The crystals were analysed by X-ray powder diffraction (XRPD). The diffractogram shows the following d-values (given in angstrom) and relative intensities: 15.2 (m), 7.6 (w), 5.1 (w), 4.12 (vw), 2.29 (vw) Å.

The significant d-values (given in angstrom) and relative intensities are: 15.2 (m), 7.6 (w), 5.1 (w), 4.12 (vw) Å.

The relative intensities (% rel.int.) are less reliable and instead of numerical values the following definitions are used:

m (medium): >50
w (weak): 15-50
vw (very weak): <15

### Example 6

### 2-Hydroxy-3-[5-(morpholin-4-ylmethyl)pyridin-2-yl]1H-indole-5-carbonitrile fumarate

2-hydroxy-3-[5-(morpholin-4-ylmethyl)pyridin-2-yl]1H-indole-5-carbonitrile (0.5765 g) was suspended in ethanol (5.7 ml) and fumaric acid (0.1893 g). The solution was heated to 40° C. for 14 hrs, and was then cooled to room temperature in 2 hrs. The crystals were filtered, washed with ethanol (2 ml) and dried in vacuum at 40° C. for 47 hrs. 0.6316 g 2-hydroxy-3-[5-(morpholin-4-ylmethyl)pyridin-2-yl]1H-indole-5-carbonitrile fumarate was obtained after drying.

¹H (500 MHz, DMSO-d₆): 14.78 (1H, br s), 10.89 (1H, s), 8.11 (1H, br s), 7.91 (1H, br s), 7.85 (1H, br d), 7.80 (1H, br dd), 7.28 (1H, dd), 7.00 (1H, d), 6.63 (2H, s), 3.59 (4H, br t), 3.42 (2H, s), 2.42 (4H, br s).

The crystals were analysed by X-ray powder diffraction (XRPD). The diffractogram shows the following d-values (given in angstrom) and relative intensities: 19.8 (w), 16.5 (m), 15.3 (w), 13.1 (m), 12.7 (m), 12.0 (w), 10.5 (w), 9.1 (w), 7.6 (w), 6.8 (m), 6.5 (w), 6.3 (w), 6.2 (w), 6.0 (m), 5.6 (w), 5.5 (w), 5.2 (w), 5.1 (w), 4.94 (w), 4.86 (m), 4.71 (w), 4.63 (w), 4.55 (m), 4.38 (m), 4.23 (w), 4.12 (w), 3.58 (m), 3.40 (m), 3.26 (s), 3.12 (m) Å.

The significant d-values (given in angstrom) and relative intensities are: 16.5 (m), 13.1 (m), 12.7 (m), 6.8 (m), 3.26 (s) Å.

The relative intensities (% rel.int.) are less reliable and instead of numerical values the following definitions are used:

s (strong): >75
m (medium): 33-75
w (weak): <33

### Example 7

### 2-Hydroxy-3-[5-(morpholin-4-ylmethyl)pyridin-2-yl]1H-indole-5-carbonitrile maleate

2-hydroxy-3-[5-(morpholin-4-ylmethyl)pyridin-2-yl]1H-indole-5-carbonitrile (0.5473 g) was suspended in ethanol (5.47 ml) and maleic acid (0.1719 g). The solution was heated to 40° C./14 hrs, and was then cooled to room temperature in 2 hrs. The crystals were filtered, washed with ethanol (2 ml) and dried in vacuum at 40° C. for 47 hrs. 0.5902 g 2-hydroxy-3-[5-(morpholin-4-ylmethyl)pyridin-2-yl]1H-indole-5-carbonitrile phosphate was obtained after drying.

¹H (500 MHz, DMSO-d₆): 14.75 (1H, br s), 10.94 (1H, s), 8.19 (1H, s), 7.96 (1H, s), 7.88 (1H, br d), 7.83 (1H, dd), 7.32 (1H, dd), 7.02 (1H, d), 6.11 (2H, s), 3.93 (2H, br s), 3.72 (4H, br s), 2.89 (4H, br s).

The crystals were analysed by X-ray powder diffraction (XRPD). The diffractogram shows the following d-values (given in angstrom) and relative intensities: 20.1 (w), 18.5 (m), 15.3 (w), 12.7 (s), 10.3 (m), 10.0 (m), 9.0 (m), 7.9 (vw), 7.6 (w), 7.4 (w), 6.8 (s), 6.5 (w), 6.3 (m), 6.1 (m), 5.6 (w), 5.3 (w), 5.2 (w), 4.78 (m), 4.67 (m), 4.58 (s), 4.46 (m), 4.36 (m), 4.23 (m), 3.98 (w), 3.79 (m), 3.14 (w), 3.05 (m), 2.94 (w) Å.

The significant d-values (given in angstrom) and relative intensities are: 12.7 (s), 6.8 (s), 6.1 (m), 4.58 (s), 3.05 (m) Å.

The relative intensities (% rel.int.) are less reliable and instead of numerical values the following definitions are used:

(strong): >50
m (medium): 14-50
w (weak): 4-14
vw (very weak): <4

Crystallinity of 2-hydroxy-3-[5-(morpholin-4-ylmethyl)pyridin-2-yl]1H-indole-5-carbonitrile citrate, mesylate, esylate, edisylate, phosphate, fumarate or maleate were analyzed using X-ray powder diffraction (XRPD) as described below:

The peaks, identified with d-values calculated from the Bragg formula and intensities, have been extracted from the diffractogram of crystalline citrate salt. Only the main peaks, that are the most characteristic, significant, distinct and/or reproducible, have been tabulated, but additional peaks can be extracted, using conventional methods, from the diffractogram. The presence of these main peaks, reproducible and within the error limit, is for most circumstances sufficient to establish the presence of said crystalline salt.

X-ray diffraction analyses were performed using a PANalytical X'Pert Pro MPD diffractometer for 64 minutes from 1 to 40° 2θ with and without internal standard reference. The 2θ angles were corrected with regard to the standard values whereafter calculation into d-values (distance values) was done. The d-values may vary in the range ±2 on the last given decimal place. The sample preparation was performed according to standard methods, for example those described in Giacovazzo, C. et al (1995), Fundamentals of Crystallography, Oxford University Press; Jenkins, R. and Snyder, R. L. (1996), Introduction to X-Ray Powder Diffractometry, John Wiley & Sons, New York; Bunn, C. W. (1948), Chemical Crystallography, Clarendon Press, London or Klug, H. P. & Alexander, L. E. (1974), X-ray Diffraction Procedures, John Wiley and Sons, New York.

The invention is described by the following example showing increased bone formation:

### Example 8

### Increased Bone Formation in Rats Treated with 2-hydroxy-3-[5-(morpholin-4-ylmethyl)pyridin-2-yl]1H-indole-5-carbonitrile

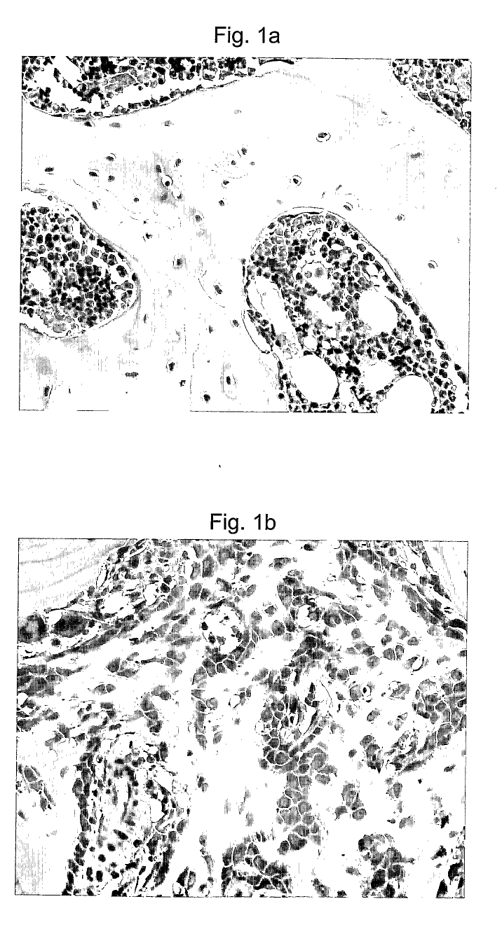

The compound 2-hydroxy-3-[5-(morpholin-4-ylmethyl)pyridin-2-yl]1H-indole-5-carbonitrile citrate was formulated as a solution in water with 0.3-5% (w/v) hydroxypropyl-β-cyclodextrine and 0.5% (w/v) ascorbic acid added. Male and female Han Wistar rats were dosed by oral gavage, at dosages of 15, 55 or 100 μmol/kg/day for four weeks and compared to vehicle controls. Complete necropsies were performed and the tissues preserved in 10% formalin. The femur, femorotibial joint and sternum were decalcified, embedded in paraffin, sectioned at 5 μm thickness and stained with hematoxylin and eosin. As evaluated by light microscopy, increased bone formation in the form of thickened trabeculae, thickened cortical bone, periosteal hyperostosis and increased number of osteoblasts occurred at the 100 μmol/kg dose level (FIG. 1b).

FIG. 1a shows normal trabeculae and bone marrow in the femur (epiphysis) of a vehicle-treated control rat. FIG. 1b shows a marked increase in formation of osteoid and increased number of osteoblasts in the same area of the femur in a rat treated with 100 μmol/kg of 2-hydroxy-3-[5-(morpholin-4-ylmethyl)pyridin-2-yl]1H-indole-5-carbonitrile for 4 weeks. (magnification: ×100).

1-11. (canceled)

12. A method of prevention and/or treatment of bone-related disorders or conditions comprising administering to a mammal in need thereof a therapeutically effective amount of 2-hydroxy-3-[5-(morpholin-4-ylmethyl)pyridin-2-yl]1H-indole-5-carbonitrile as a free base or a pharmaceutically acceptable salt thereof.

13. A method of prevention and/or treatment of bone-related disorders comprising administering to a mammal in need thereof a therapeutically effective amount of 2-hydroxy-3-[5-(morpholin-4-ylmethyl)pyridin-2-yl]1H-indole-5-carbonitrile citrate.

14. A method of prevention and/or treatment of bone-related disorders according to claim 12, comprising administering to a mammal in need thereof a therapeutically effective amount of a salt selected from
2-hydroxy-3-[5-(morpholin-4-ylmethyl)pyridin-2-yl]1H-indole-5-carbonitrile mesylate,
2-hydroxy-3-[5-(morpholin-4-ylmethyl)pyridin-2-yl]1H-indole-5-carbonitrile esylate,
2-hydroxy-3-[5-(morpholin-4-ylmethyl)pyridin-2-yl]1H-indole-5-carbonitrile edisylate,
2-hydroxy-3-[5-(morpholin-4-ylmethyl)pyridin-2-yl]1H-indole-5-carbonitrile phosphate,
2-hydroxy-3-[5-(morpholin-4-ylmethyl)pyridin-2-yl]1H-indole-5-carbonitrile fumarate or
2-hydroxy-3-[5-(morpholin-4-ylmethyl)pyridin-2-yl]1H-indole-5-carbonitrile maleate.

15. A method of prevention and/or treatment of osteoporosis comprising administering to a mammal a therapeutically effective amount of a compound as described in claim 12.

16. A method of increasing bone formation comprising administering to a mammal a therapeutically effective amount of a compound as described in claim 12.

17. A method of increasing cancellous bone formation and/or new bone formation comprising administering to a mammal a therapeutically effective amount of a compound as described in claim 12.

18. A method of increasing bone mineral density comprising administering to a mammal a therapeutically effective amount of a compound as described in claim 12.

19. A method of reducing the incidence of fracture comprising administering to a mammal a therapeutically effective amount of a compound as described in claim 12.

20. A method of enhancing fracture healing comprising administering to a mammal, a therapeutically effective amount of a compound as described in claim 12.

21-22. (canceled)

23. A method according to claim 15 wherein said mammal is human.

24. A method according to claim 16 wherein said mammal is human.

25. A method according to claim 17 wherein said mammal is human.

26. A method according to claim 18 wherein said mammal is human.

27. A method according to claim 19 wherein said mammal is human.

28. A method according to claim 20 wherein said mammal is human.
